(19) United States
(12) Patent Application Publication
(10) Pub. No.: US 2010/0016893 A1
(43) Pub. Date: Jan. 21, 2010

(54) METHOD AND APPARATUS FOR ENHANCING THE FIXATION OF BONE AND SOFT TISSUE ANCHORS

(76) Inventor: Gary S. Fanton, Portola Valley, CA (US)

Correspondence Address: Mark J. Pandiscio;Pandiscio & Pandiscio, P. C., 470 Totten Pond Road, Waltham, MA, 02451-1914, US

(21) Appl. No.: 12/398,796

(22) Filed: Mar. 5, 2009

Related U.S. Application Data
(60) Provisional application No. 61/068,208, filed on Mar. 5, 2008.

(51) Int. Cl. A61B 17/04

(52) U.S. Cl. 606/232

(57) ABSTRACT

Apparatus for enhancing the fixation of an anchor in a body part, wherein the body part comprises an interior portion and an exterior surface, and wherein the anchor is disposed within the interior portion of the body part, the apparatus comprising:
a fixation enhancement plug for disposition within the body part, wherein the fixation enhancement plug is disposed between the anchor and the exterior surface of the body part, and further wherein the fixation enhancement plug is configured to block the anchor from exiting the exterior surface of the body part.

A method for enhancing the fixation of an anchor in a body part, wherein the body part comprises an interior portion and an exterior surface, and wherein the anchor is disposed within the interior portion of the body part, the method comprising:
deploying a fixation enhancement plug within the body part so that the fixation enhancement plug is disposed between the anchor and the exterior surface of the body part, so that the fixation enhancement plug blocks the anchor from exiting the exterior surface of the body part.

### REFERENCE TO PENDING PRIOR PATENT APPLICATION

This patent application claims benefit of pending prior U.S. Provisional Patent Application Ser. No. 61/068,208, filed Mar. 5, 2008 by Gary S. Fanton for METHOD AND APPARATUS FOR SECURING FIXATION OF BONE AND SOFT TISSUE ANCHORS (Attorney's Docket No. FANTON-1 PROV), which patent application is hereby incorporated herein by reference.

### FIELD OF THE INVENTION

This invention relates to surgical methods and apparatus in general, and more particularly to surgical methods and apparatus for attaching objects to bone and soft tissue.

### BACKGROUND OF THE INVENTION

Many attempts have been made to provide devices that allow the securing of soft tissue to a human bone. For example, numerous devices have been designed to allow a torn rotator cuff to be secured to the humeral head of the shoulder. These devices are typically inserted during a surgical procedure conducted through an open incision or by arthroscopic means.

Typically, in a first step, a hole is drilled in the bone under arthroscopic or open incision visualization. A length of suture is secured to the anchor and threaded through a portion of the soft tissue which is to be secured to the bone.

Once the suture is coupled between the soft tissue and the bone anchor, the bone anchor may be inserted into the hole that was drilled in the bone. In some designs, the bone anchor may be configured to lock itself within the bone hole upon deployment of a locking mechanism. In other designs, the bone anchor may be configured to lock itself within the bone based on the geometry of the bone anchor, without requiring the deployment of a locking mechanism.

In still other configurations, the bone anchor may be impacted into the bone without requiring a hole to be pre-drilled in the bone.

Thus it will be appreciated that several means of securing the bone anchor within a hole in the bone are known in the art.

Various methods for securing the suture to the bone anchor, and for securing the suture to the soft tissue, are also known in the art. For example, some bone anchor designs allow the surgeon to tie a knot in the length of suture adjacent to the soft tissue so as to secure the soft tissue to the bone. In other bone anchor designs, the length of suture can be adjustably locked within or around the bone anchor, allowing for knotless fixation of the suture to the bone anchor. Thus, it will be seen that the length of suture securing the soft tissue to the bone can be adjusted at either the soft tissue side or the bone anchor side. In any case, however, once the bone anchor is locked into the bone, the bone anchor cannot be unlocked or adjusted in position after the suture length is fixed to the soft tissue.

It is highly disadvantageous for the soft tissue to migrate away from the bone surface, either at the time of repair and/or during the healing process. Approximation of the soft tissue to bone (especially muscle, tendon and ligament tissue) typically results in tension being applied to the suture and the bone anchor, either through the inherent retraction forces of the tissue and/or through movement of body parts. In an ideal surgical setting, a bone anchor is locked within the bone firmly, with sufficient fixation to overcome the pull of the secured soft tissue.

It is well known in the medical literature that ideal settings for bone anchor fixation often do not exist. Factors that relate to compromised bone anchor fixation include, but are not limited to, (i) regions of soft bone, (ii) diseased bone, (iii) previously-operated-on bone, and (iv) bone holes that are made too large for proper bone anchor fixation. In these and other settings, the distraction forces of the soft tissue may overcome the holding power of the bone anchor and result in bone anchor migration toward the surface of the bone and, in some circumstances, even allow the bone anchor to migrate completely out of the bone hole. In such circumstances, adequate fixation of the tissue to bone may be jeopardized or completely lost.

Thus, it would be advantageous, in conditions that might result (or have resulted) in bone anchor migration, to improve the fixation of a previously-deployed bone anchor, without sacrificing the strength of the attached length of suture.

In addition to the foregoing, it has been found that, after soft tissue fixation with a bone anchor, a suture length may stretch, or a suture knot may slip, as a result of the retraction forces applied to the suture by the soft tissue, again jeopardizing secure tissue to bone approximation.

And in still other settings, it has been found that the soft tissue integrity may be insufficient to adequately hold the suture in place within the tissue, thereby allowing migration of the soft tissue away from the bone.

In these, and other, circumstances, it would be advantageous to provide a means for advancing a previously-set bone anchor deeper into the bone, thus taking up any slack in the suture and improving approximation of the soft tissue to bone.

In still other situations, it may be desirable to secure an object to soft tissue. In these situations, a soft tissue anchor may be used to secure the object to the soft tissue, in a manner analogous to the bone anchor fixation discussed above. The aforementioned problems of anchor migration, and/or suture stretching and/or knot slippage, and/or suture movement within the object being secured, can occur in these situations as well.

Thus, it would be advantageous, in conditions that might result (or have resulted) in anchor migration, to improve the fixation of a previously-deployed soft tissue anchor, without sacrificing the strength of the attached length of suture.

And it would be advantageous to provide a means for advancing a previously-set soft tissue anchor deeper into the tissue, thus taking up any slack in the suture and improving approximation of the object to tissue.

### SUMMARY OF THE INVENTION

The present invention addresses one or more of the problems discussed above by providing a novel method and apparatus for enhancing the fixation of bone and soft tissue anchors. More particularly, the present invention discloses a novel method and apparatus for enhancing anchor fixation by “backing up” a previously-placed anchor with an additional fixation element so as to prevent migration of the previously-placed anchor. This additional fixation element may comprise a fixation enhancement plug, wherein the plug may comprise (i) a pre-manufactured device deployed into the bone hole or tissue proximal to the previously-placed anchor, and/or (ii) a flowable mass which is deployed into the bone hole or tissue proximal to the previously-placed anchor and then solidified in situ.

The present invention also provides a novel method and apparatus that can advance a previously-deployed anchor deeper into the bone or tissue so as to take up any slack in a length of suture (or sutures) attached to a previously-deployed anchor, as well as providing enhanced fixation for the previously-deployed anchor once it has been set to its desired depth.

And the present invention provides a novel method and apparatus for providing enhanced anchor fixation by, optionally, stacking a plurality of fixation enhancement plugs between the previously-deployed anchor and the proximal portion of the bone or tissue hole.

Furthermore, the present invention provides a novel method and apparatus for enhanced anchor fixation, wherein the enhanced anchor fixation is designed to minimize trauma to a previously-placed suture (or sutures).

The present invention also comprises a novel method and apparatus for enhanced anchor fixation in bone, wherein biological material, such as bone, is used to enhance healing and bone grafting of the bone hole.

### BRIEF DESCRIPTION OF THE DRAWINGS

These and other objects and features of the present invention will be more fully disclosed or rendered obvious by the following detailed description of the invention, which is to be considered together with the accompanying drawings wherein like numbers refer to like parts, and further wherein:

FIG. 1 is a schematic view showing a bone anchor deployed in a bone hole and securing soft tissue to the bone;

FIG. 2 is a schematic view showing how soft tissue can pull the bone anchor proximally when the bone anchor is deployed in soft bone;

FIG. 3 is a schematic view showing how the soft tissue can retract from its desired approximation site when the bone anchor backs out of the bone hole;

FIGS. 4-7 are schematic views showing how a fixation enhancement plug can be inserted into the bone hole proximal to the bone anchor so as to re-seat a migrated bone anchor and thereafter hold the bone anchor in position within the bone hole, with the fixation enhancement plug being in the form of a pre-manufactured screw thread device;

FIG. 8 is a schematic view showing how a fixation enhancement plug, in the form of a pre-manufactured expandable device, can be used to enhance the fixation of a bone anchor;

FIG. 9 is a schematic view showing how a fixation enhancement plug, in the form of a pre-manufactured winged device, can be used to enhance the fixation of a bone anchor;

FIG. 10 is a schematic view showing how a fixation enhancement plug, in the form of a flowable mass solidified in situ, can be used to enhance the fixation of a bone anchor;

FIGS. 11-14 are schematic views showing alternative forms of fixation enhancement plugs;

FIGS. 15 and 16 are schematic views showing how the fixation enhancement plug can be used to drive a migrated bone anchor back into place;

FIG. 17 is a series of schematic views showing tissue being attached to bone using (i) an exemplary bone anchor, and (ii) a fixation enhancement plug formed in accordance with the present invention;

FIG. 18 is a series of schematic views showing another form of the invention, where the bone anchor may be completely eliminated, and only the fixation enhancement plug and its associated inserter used to attach soft tissue to bone; and

FIG. 19 is a schematic view showing an alternative form of inserter tool, where the distal end of the inserter tool comprises an open-ended slot rather than an eyelet.

### DETAILED DESCRIPTION OF THE PREFERRED EMBODIMENTS

The present invention discloses a novel method and apparatus for enhancing anchor fixation by “backing up” a previously-placed anchor with an additional fixation element so as to prevent migration of the previously-placed anchor. This additional fixation element may comprise a fixation enhancement plug, wherein the plug may comprise (i) a pre-manufactured device deployed into the bone hole or tissue proximal to the previously-placed anchor, and/or (ii) a flowable mass which is deployed into the bone hole or tissue proximal to the previously-placed anchor and then solidified in situ.

The present invention also provides a novel method and apparatus that can advance a previously-deployed anchor deeper into the bone or tissue so as to take up any slack in a length of suture (or sutures) attached to a previously-deployed anchor, as well as providing enhanced fixation for the previously-deployed anchor once it has been set to the desired depth.

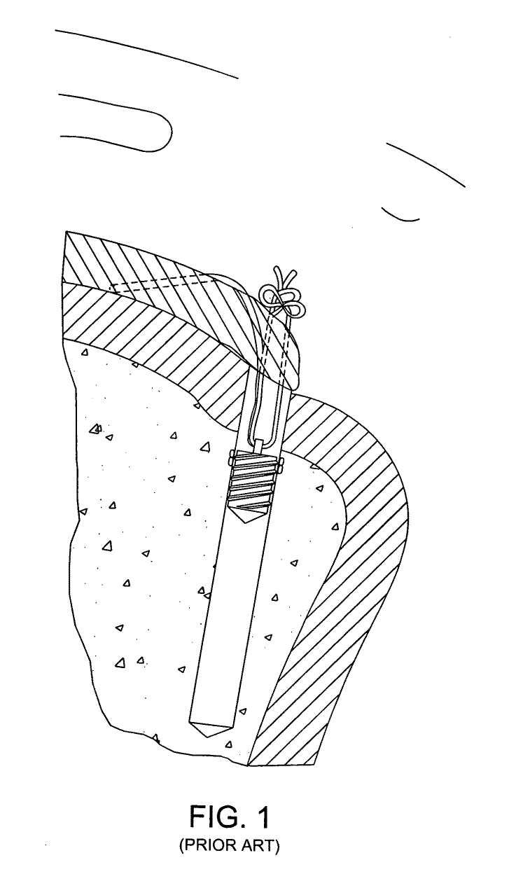

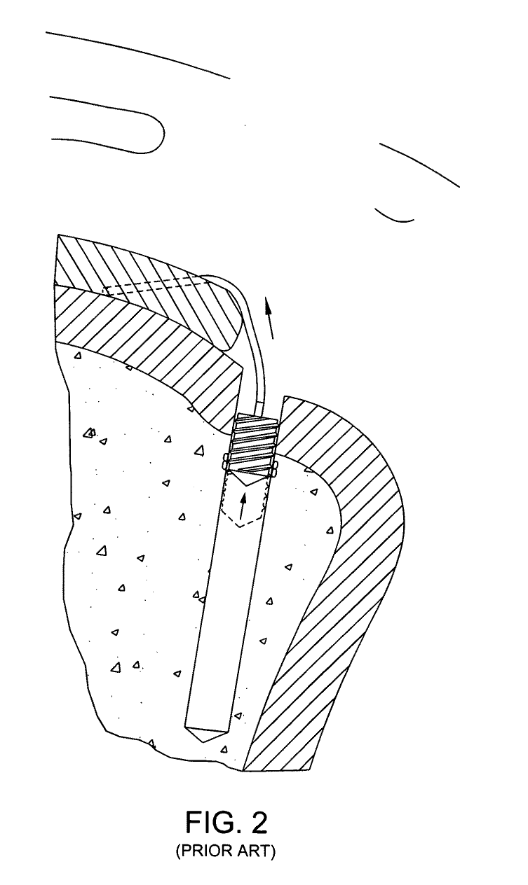

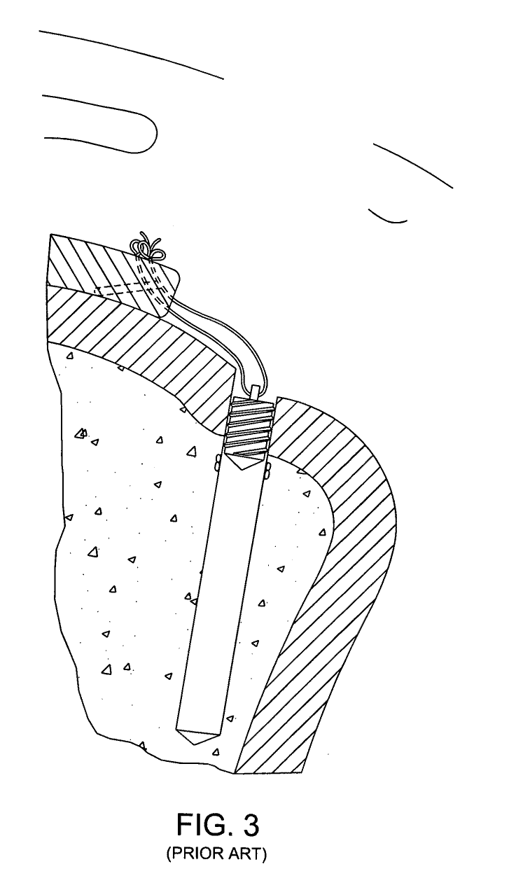

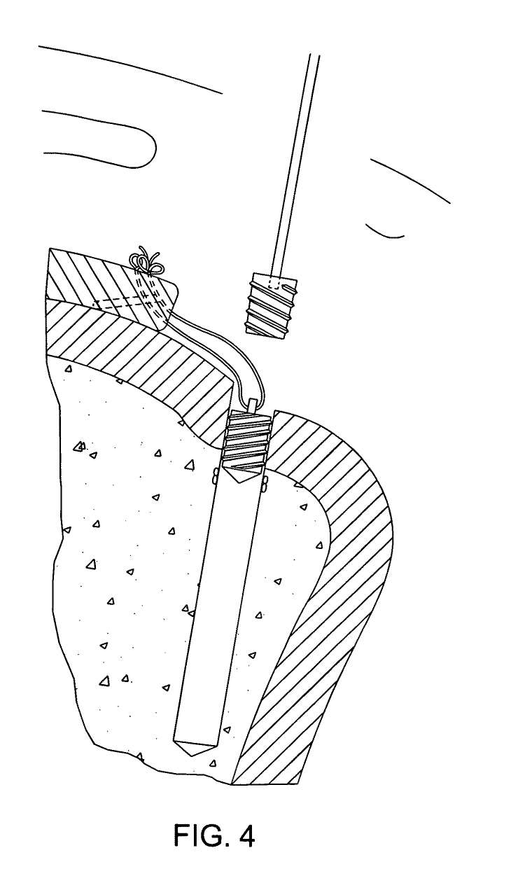

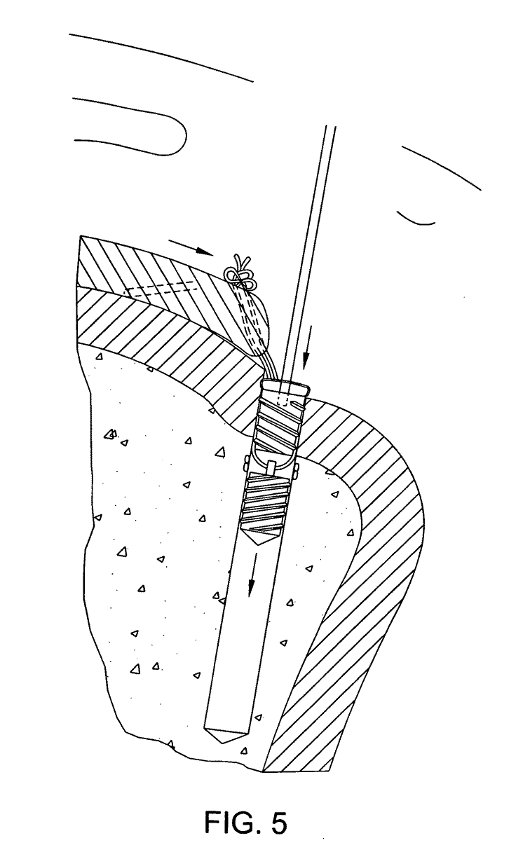

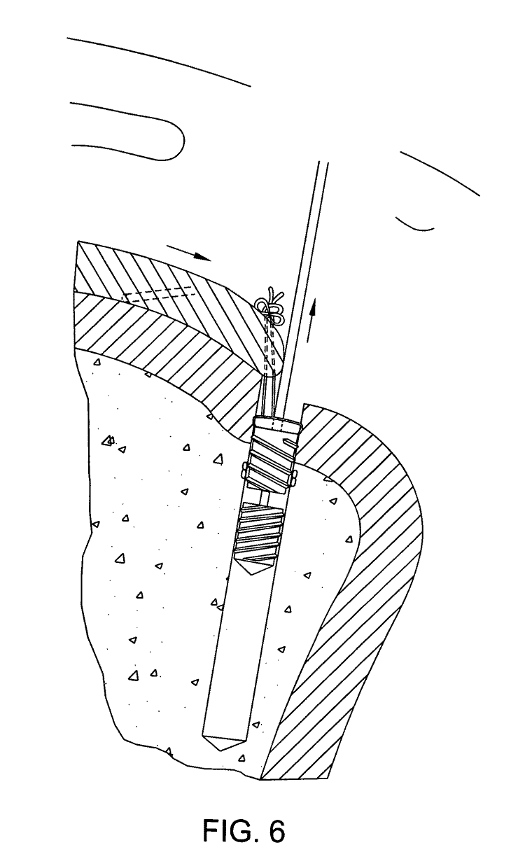

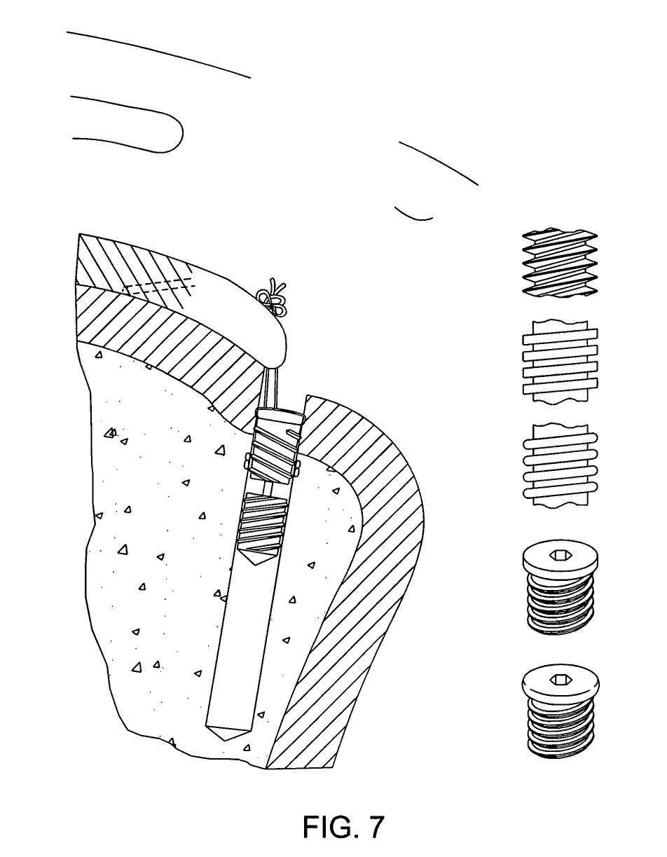

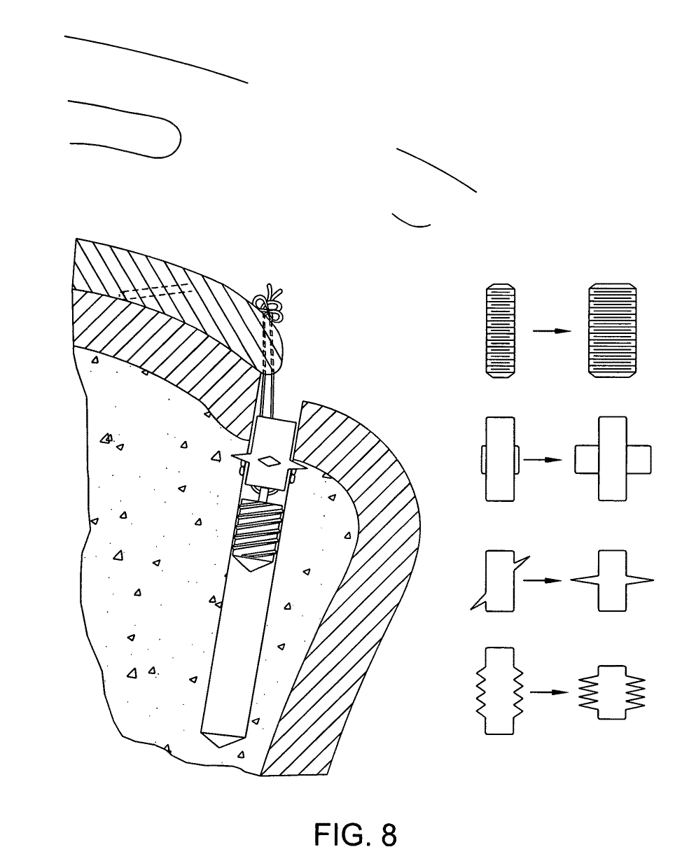

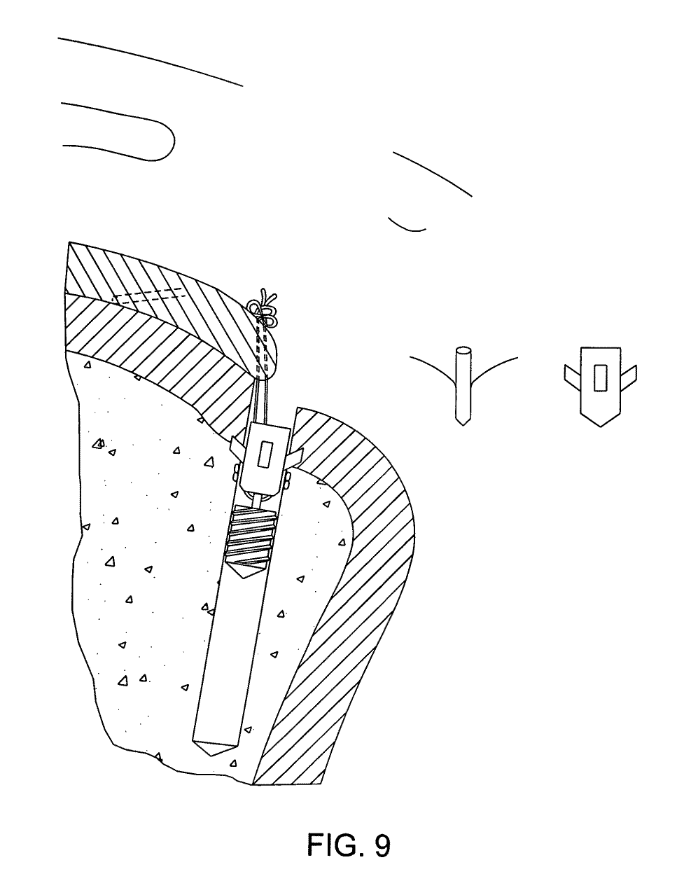

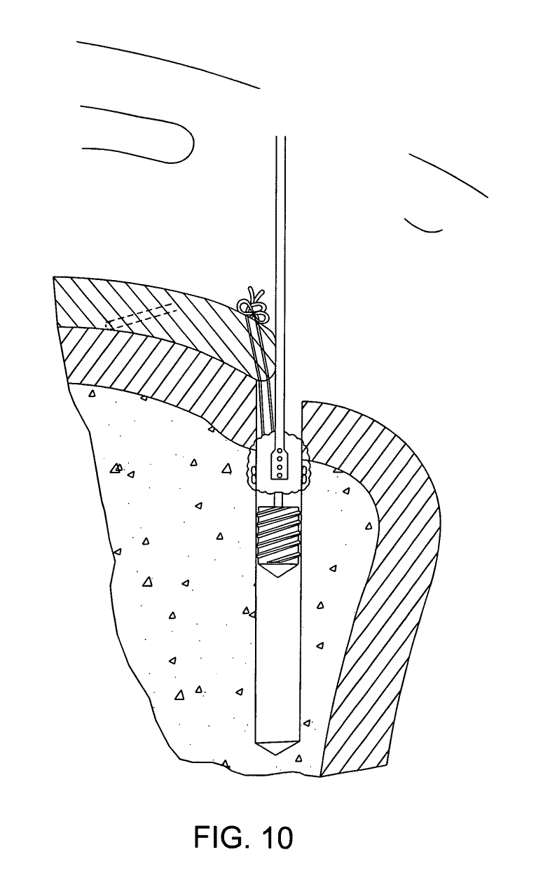

The present invention may be incorporated in many different embodiments without departing from the scope of the invention. By way of example but not limitation, and looking now at FIGS. 1-10, the present invention can utilize one or more of the following constructions:

1. threaded fixation enhancement plug, which:
a. can be made from metals, synthetics, biocompatibles, bioabsorbables, and/or bone materials;
b. can be a solid and/or cannulated body;
c. can be formed in a range of different diameters and/or lengths;
d. can utilize a sharp thread design and/or a non-cutting thread design (e.g., rounded threads, flat threads, etc.);
e. can utilize a “headless” design to fit flush with bone hole;
f. can utilize a rounded head to compress into bone hole;
g. can be tapered vs. cylindrical;
h. can have a flat distal surface to abut or push against a previously-placed anchor; and/or
i. may be screwed into place; and/or

2. expandible fixation enhancement plug, which:
a. can be made from metals, synthetics, biocompatibles, bioabsorbables, and/or bone materials;
b. can be in various shapes (e.g., cylindrical, round, disc-shaped, plurality of flat surfaces to form a multi-sided structure, etc.);
c. deployed into bone hole with insertion tool; and/or
d. can be solid and/or cannulated; and/or

3. winged fixation enhancement device, which:
a. can be made from metals, synthetics, biocompatibles, bioabsorbables, and/or bone materials;
b. can utilize mechanical deployment vs. self-deploying mechanisms (e.g., expanded wings);
c. can have ring-shaped wings vs. a plurality of separate wings;
d. can utilize barbed wings; and/or
e. can be pushed vs. pounded into bone hole; and/or

4. deployment of epoxies, glues, and/or cements to create the fixation enhancement plug in-situ (i.e., the bone anchor can be “backed up” by forming the fixation enhancement plug in-situ, e.g., out of epoxies, glues and/or cements—in this case, the epoxy, glue and/or cement is flowed into place behind the previously-deployed anchor and then the epoxy, glue and/or cement is cured or hardened in-situ so as to create the solid mass which constitutes the fixation enhancement plug).

The present invention also provides a novel method and apparatus for providing enhanced anchor fixation by stacking a plurality of fixation enhancement plugs between the previously-deployed anchor and the proximal portion of the bone or tissue hole.

And the present invention provides a novel method and apparatus for enhanced anchor fixation, wherein the enhanced anchor fixation is designed to minimize trauma to a previously-placed suture (or sutures).

The present invention also comprises a novel method and apparatus for enhanced anchor fixation in bone, wherein biological material, such as bone, is used to enhance healing and bone grafting of the bone hole.

### Further Aspects Of The Invention

It will be appreciated that the present invention comprises, among other things:

1. methods of enhanced fixation;
a. stacked fixation of bone anchor with plug;
b. stacked fixation and advancement of bone anchor with plug;
c. can be used for primary cases as well as revision surgery; and/or
d. can be used in various conditions of use (soft bone vs. hole too big vs. laxity of sutures and/or knot, etc.); and/or

2. devices, e.g.:
a. screw with various designs, preferably with flat end and non-cutting threads;
b. expandible devices that can be pushed or hammered into hole and compress suture but avoid shear;
c. specifically-designed, variable size winged plugs that are tapped into hole; and/or
d. epoxies, glues and/or cements, etc. that will set in the bone hole so as to form the fixation enhancement plug in situ, possibly expanding in size while setting.

### Additional Concepts

Where the fixation enhancement plug has a screw-type configuration (i.e., where the fixation enhancement plug it is set by screwing the fixation enhancement plug into the bone), it is also possible to form the screw-in enhancement plug as a 2-part screw. More particularly, in this construction, the deepest part of the fixation enhancement plug is preferably a flat or blunt end that swivels relative to the proximal threaded part. This construction permits the distal end of the fixation enhancement plug to engage the bone anchor while preventing the screw-in enhancement plug from grabbing and/or twisting the embedded bone anchor within the hole as the enhancement plug is advanced by turning. The use of a swivel hinge eliminates torque on the embedded anchor.

### Additional Subject Matter

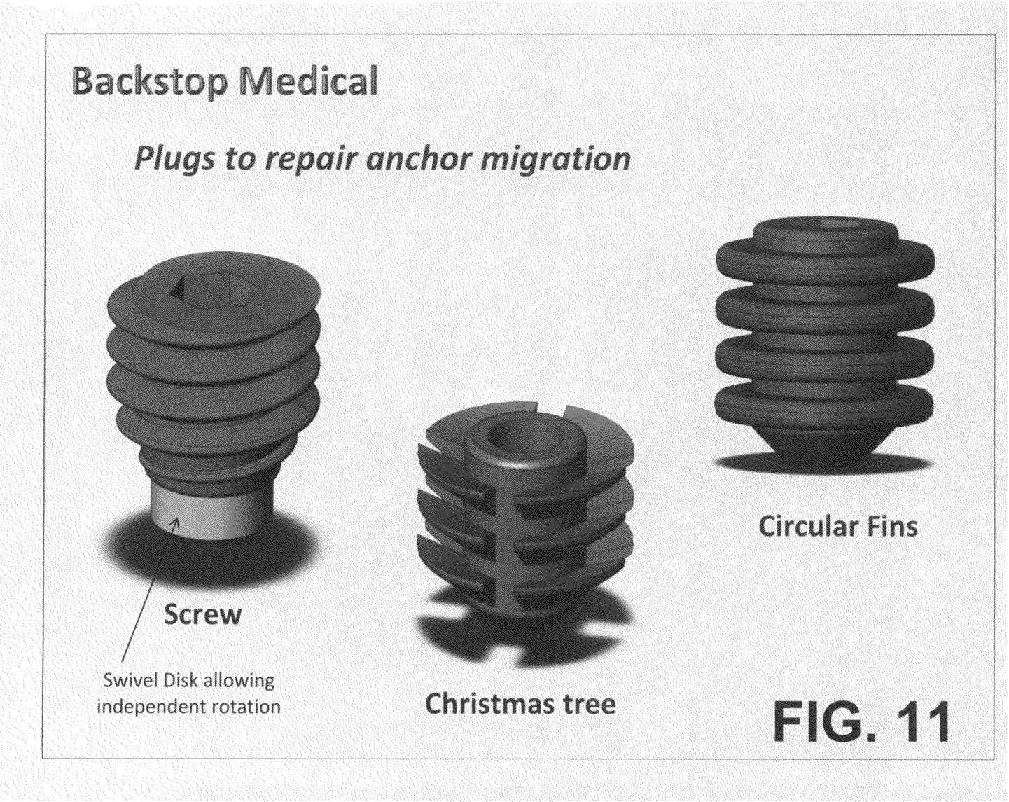

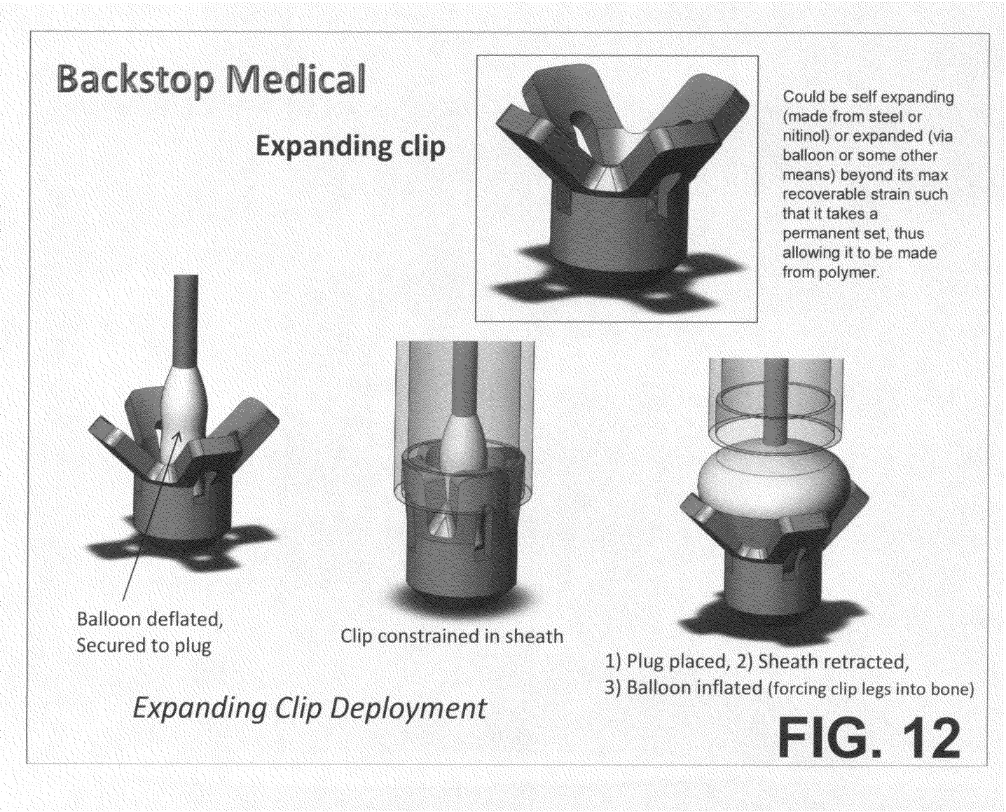

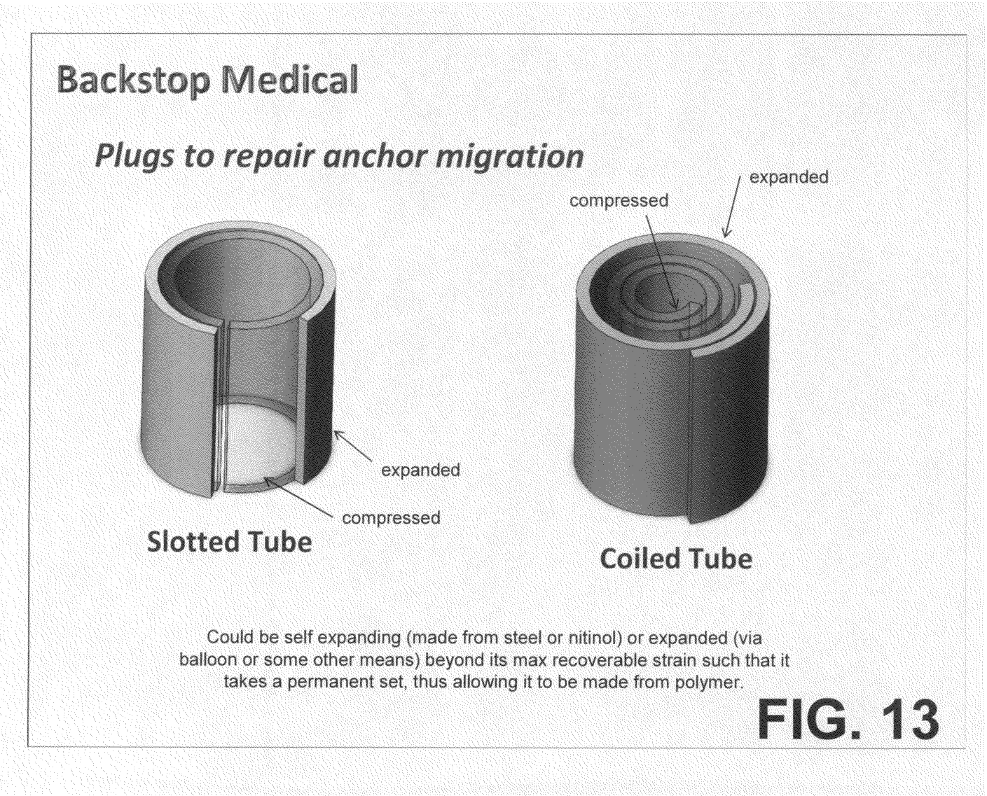

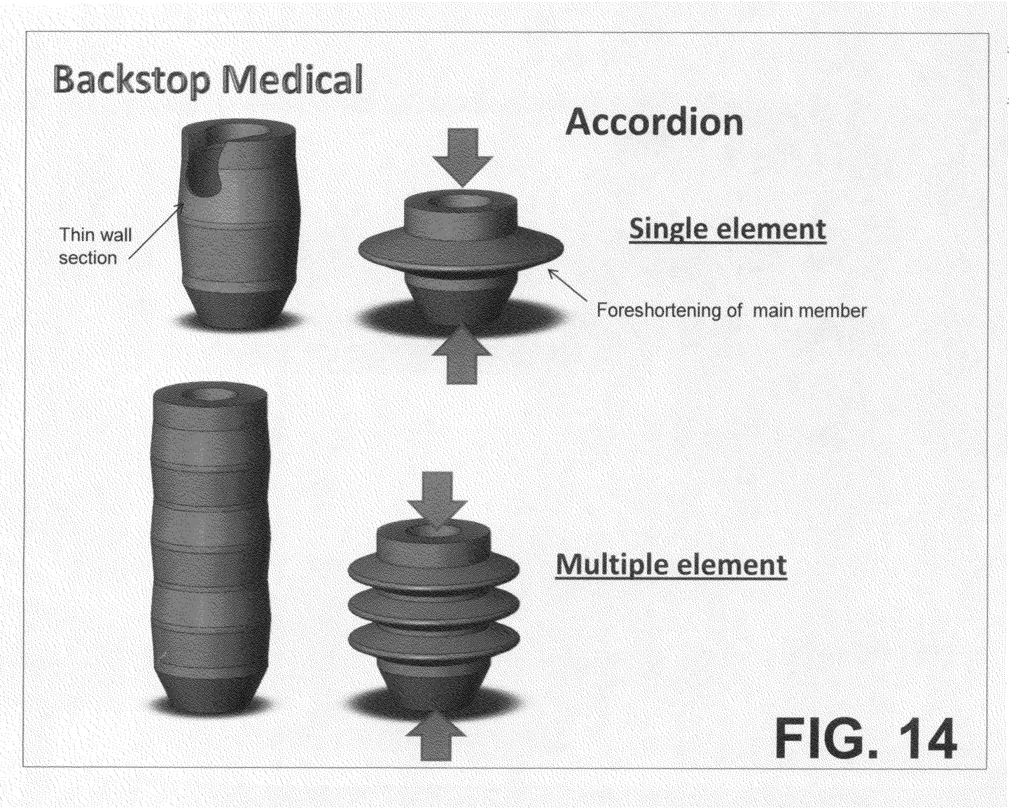

See also the attached FIGS. 11-14, which show construction details of some preferred forms of the fixation enhancement plug. Note that where the fixation enhancement plug comprises a screw-in device, the screw threads are preferably formed as non-cutting threads so as to avoid cutting or damaging the suture. Note also that the fixation enhancement plug may have a coated or lubricated surface so as to further minimize suture abrasion.

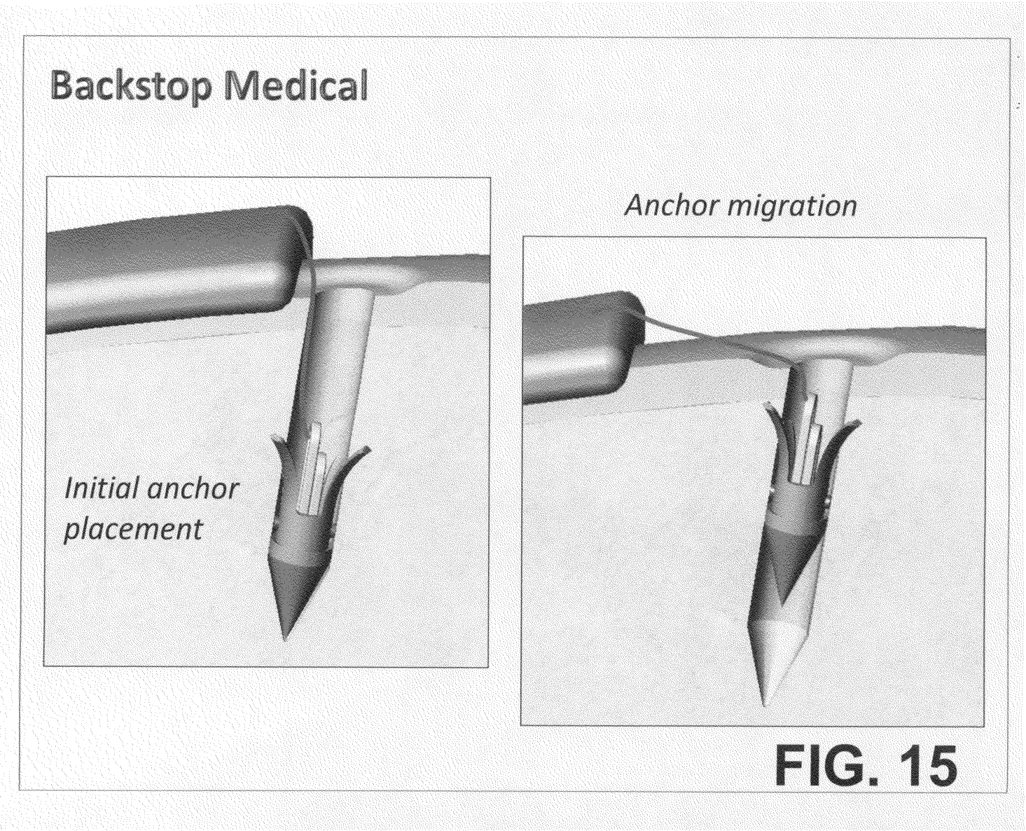

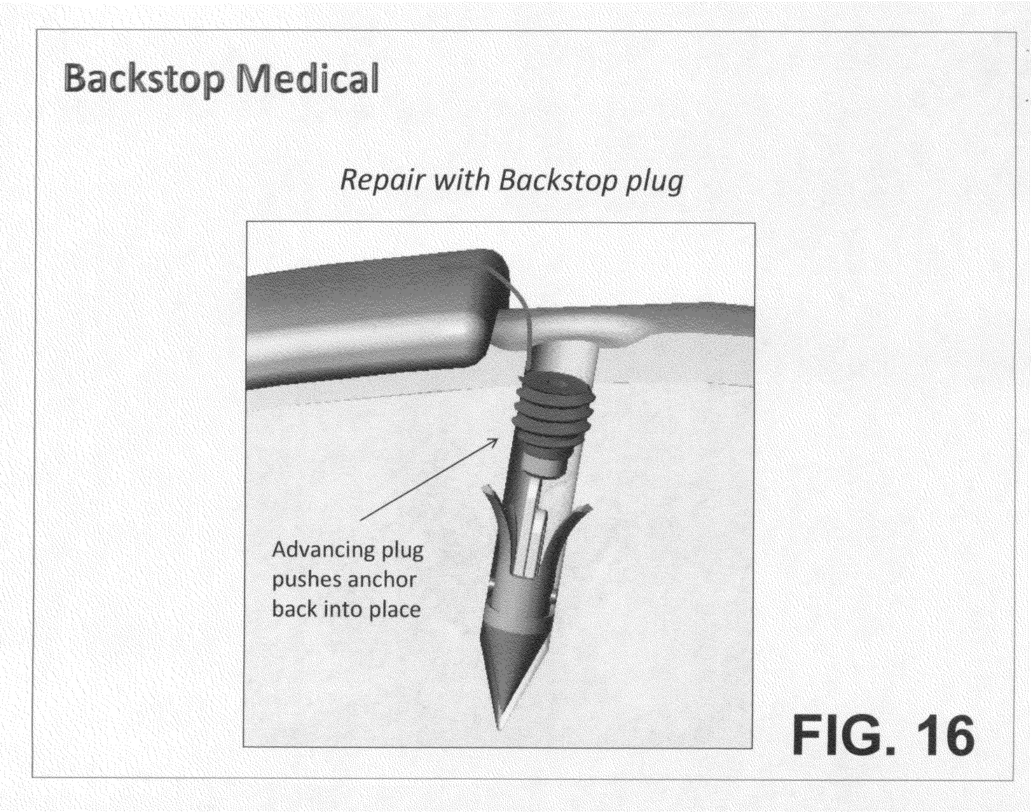

See also FIGS. 15 and 16, which show anchor migration (FIG. 15) and how the fixation enhancement plug can drive a migrated anchor back into place (FIG. 16).

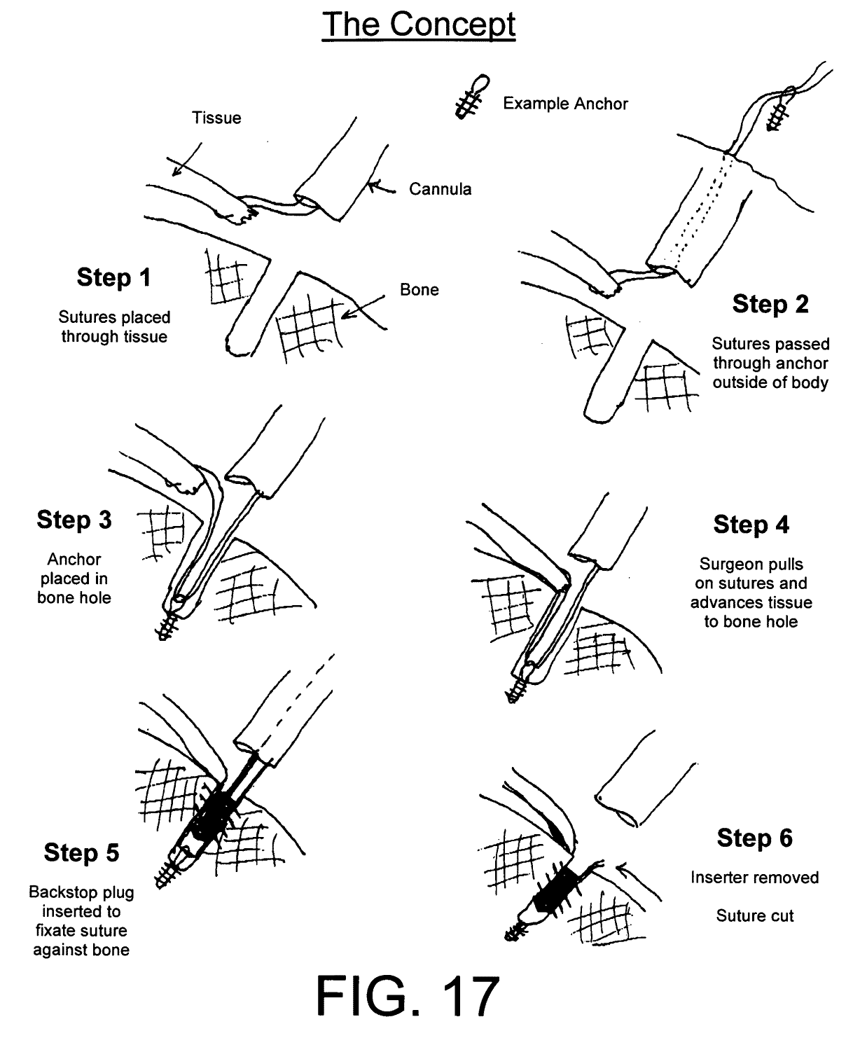

FIG. 17 is a series of schematic views showing tissue being attached to bone using (i) an exemplary bone anchor, and (ii) the fixation enhancement plug of the present invention. In this respect it should be appreciated that, while the exemplary bone anchor shown in FIG. 17 appears to have the general structure of a screw-in anchor or a push-in anchor, it should be understood that the exemplary bone anchor can be substantially any bone anchor, e.g., a barb-type bone anchor.

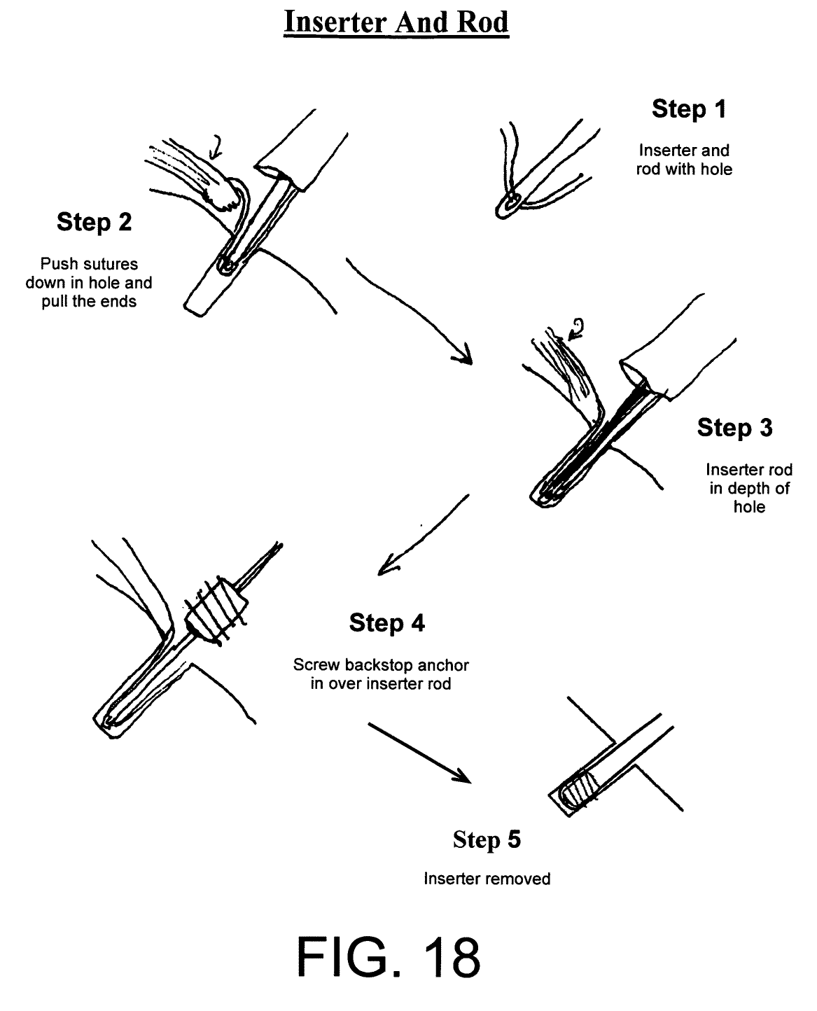

FIG. 18 is a series of schematic views showing another form of the invention where the bone anchor may be completely eliminated, and only the fixation enhancement plug and its associated inserter used to attach the tissue to bone. More particularly, in this form of the invention, a special inserter tool having an eyelet on its distal end is first used to draw the suture down into the bone hole. Then the fixation enhancement plug is advanced down into the bone hole (e.g., by moving it distally along the inserter tool) so as to capture the suture against the side wall of the bone hole. Then the inserter tool is withdrawn, leaving the fixation enhancement plug binding the suture to the side wall of the bone hole.

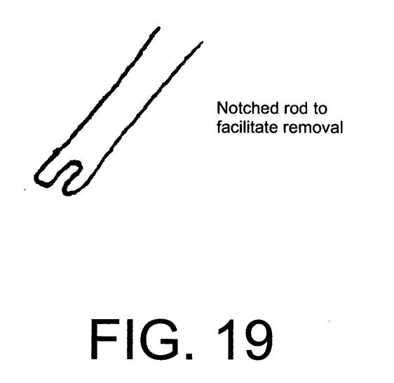

FIG. 19 shows an alternative form of inserter tool, where the distal end of the inserter tool comprises an open-ended slot rather than an eyelet. This construction is believed to facilitate release of the suture from the inserter tool after the fixation enhancement plug has been fixed in the bone hole.

Thus, in one preferred form of the present invention, the following steps may be used to secure soft tissue to bone:

1. the suture is placed through the tissue;

2. the suture is either (i) placed down the bone hole with a separate bone anchor, or (ii) held in the bone hole with an inserter tool;

3. the surgeon pulls on the suture limbs, either manually or mechanically to advance the tissue to the bone; and

4. the fixation enhancement plug is positioned in the hole (e.g., screwed in, tapped in, inserted and expanded, etc.)—in step 2(ii) above, the fixation enhancement device can be advanced into the bone hole over the inserter tool.

### Modifications of the Preferred Embodiments

It will be appreciated that still further embodiments of the present invention will be apparent to those skilled in the art in view of the present disclosure. It is to be understood that the present invention is by no means limited to the particular constructions and method steps herein disclosed and/or shown in the drawings, but also comprises any modifications or equivalents within the scope of the invention.

What is claimed is:

1. Apparatus for enhancing the fixation of an anchor in a body part, wherein the body part comprises an interior portion and an exterior surface, and wherein the anchor is disposed within the interior portion of the body part, the apparatus comprising:
a fixation enhancement plug for disposition within the body part, wherein the fixation enhancement plug is disposed between the anchor and the exterior surface of the body part, and further wherein the fixation enhancement plug is configured to block the anchor from exiting the exterior surface of the body part.

2. A method for enhancing the fixation of an anchor in a body part, wherein the body part comprises an interior portion and an exterior surface, and wherein the anchor is disposed within the interior portion of the body part, the method comprising:
deploying a fixation enhancement plug within the body part so that the fixation enhancement plug is disposed between the anchor and the exterior surface of the body part, so that the fixation enhancement plug blocks the anchor from exiting the exterior surface of the body part.

3. A system for anchoring suture to a body part, wherein the body part comprises an interior portion and an exterior surface, the system comprising:
an anchor having at least one length of suture extending therefrom, wherein the anchor is configured to be disposed within the interior portion of the body part so that the suture extends from the anchor, through the intervening portions of the body part, and exits the exterior surface of the body part; and
a fixation enhancement plug for disposition within the body part, wherein the fixation enhancement plug is disposed between the anchor and the exterior surface of the body part, and further wherein the fixation enhancement plug is configured to block the anchor from exiting the exterior surface of the body part.

4. A system according to claim 3 wherein the fixation enhancement plug comprises a pre-manufactured device.

5. A system according to claim 3 wherein the pre-manufactured device comprises screw threads.

6. A system according to claim 3 wherein the pre-manufactured device comprises an expandable structure.

7. A system according to claim 3 wherein the pre-manufactured device comprises wings.

8. A system according to claim 3 wherein the fixation enhancement plug comprises a flowable mass which is solidified in-situ.

9. A system according to claim 8 wherein the flowable mass comprises at least one from the group consisting of epoxy, glue and cement.

10. A system according to claim 3 further comprising a second fixation enhancement plug disposed between the fixation enhancement plug and the exterior surface of the body part.

11. A system according to claim 3 wherein the body part comprises bone.

12. A system according to claim 3 wherein the body part comprises tissue other than bone.

13. A method for anchoring suture to a body part, wherein the body part comprises an interior portion and an exterior surface, the method comprising:
providing a system comprising:
an anchor having at least one length of suture extending therefrom, wherein the anchor is configured to be disposed within the interior portion of the body part so that the suture extends from the anchor, through the intervening portions of the body part, and exits the exterior surface of the body part; and
a fixation enhancement plug for disposition within the body part, wherein the fixation enhancement plug is disposed between the anchor and the exterior surface of the body part, and further wherein the fixation enhancement plug is configured to block the anchor from exiting the exterior surface of the body part;
deploying the anchor in the interior portion of the body part; and
deploying the fixation enhancement plug within the body part, between the anchor and the exterior surface of the body part.

14. A system according to claim 13 wherein the fixation enhancement plug comprises a pre-manufactured device.

15. A system according to claim 13 wherein the pre-manufactured device comprises screw threads.

16. A system according to claim 13 wherein the pre-manufactured device comprises an expandable structure.

17. A system according to claim 13 wherein the pre-manufactured device comprises wings.

18. A system according to claim 13 wherein the fixation enhancement plug comprises a flowable mass which is solidified in-situ.

19. A system according to claim 18 wherein the flowable mass comprises at least one from the group consisting of epoxy, glue and cement.

20. A system according to claim 13 further comprising a second fixation enhancement plug disposed between the fixation enhancement plug and the exterior surface of the body part.

21. A system according to claim 13 wherein the body part comprises bone.

22. A system according to claim 13 wherein the body part comprises tissue other than bone.
